(19) United States
(12) Patent Application Publication
(10) Pub. No.: US 2013/0172664 A1
(43) Pub. Date: Jul. 4, 2013

(54) DEVICE FOR SUPPORTING THE EMPTYING OF THE BLADDER OF A PATIENT AND METHOD FOR OPERATING SUCH A DEVICE

(71) Applicants: Thomas Schmid, Starnberg (DE); Hiep T. Nguyen, Boston, MA (US)

(72) Inventors: Thomas Schmid, Starnberg (DE); Hiep T. Nguyen, Boston, MA (US)

(73) Assignee: DUALIS MedTech GmbH and Childrens Hospital Boston Dept. of Urology

(21) Appl. No.: 13/341,305

(22) Filed: Dec. 30, 2011

(51) Int. Cl. A61F 2/02

(52) U.S. Cl. 600/30

(57) ABSTRACT

A device for supporting the emptying of the bladder of a patient, the device comprising: an essentially stiff dome-shaped surrounding element for surrounding at least a part of the bladder having a variable volume, the device comprising at least one sensor for detecting the filling level of the bladder, the surrounding element being connectable with the bladder at a lower end of the surrounding element in a fluid-tight manner, so that an inner reservoir is defined in a hollow space between the surrounding element and the wall of the bladder.

### BACKGROUND

1. Field of the Invention

The disclosure is related to a device for supporting the emptying of the bladder of a patient and further to a method for operating such a device.

2. Discussion of the Background Art

Paraplegic patients may have the problem that an autonomous emptying of their bladder is not possible. Further, such patients may be unable to detect the filling level of their bladder.

It is an object of the present disclosure to provide a device for supporting the emptying of the bladder of a patient and a method for operating such a device.

### SUMMARY

The disclosed device comprises an essentially stiff dome-shaped surrounding element for surrounding at least a part of the bladder. E.g. the surrounding element can surround the part of the bladder which has a variable volume. This surrounding element is essentially stiff meaning that at least a part thereof is stiff or inelastic, e.g., the upper part of this surrounding element may be stiff or inelastic, whereas the lower end of the surrounding element facing towards the bladder may be elastic, so that the surrounding element is partially elastic.

The disclosed device further comprises at least one sensor for detecting the filling level of the bladder. Such a sensor can be placed, e.g., at the inner side of the surrounding element facing towards the bladder or on the bladder wall itself.

The surrounding element is connectable with the bladder at a lower end of the surrounding element in a fluid-tight manner so that an inner reservoir is defined in a hollow space between the surrounding element and the wall of the bladder.

The disclosed device further comprises a flexible membrane surrounding the surrounding element. It is preferred that this flexible outer membrane is inelastic. This flexible membrane is connectable with the bladder in a fluid-tight manner so that an outer reservoir is defined in a hollow space between the flexible membrane and the surrounding element. Thus, there are two reservoirs, a first inner reservoir inside the surrounding element and a second outer reservoir outside the surrounding element which are separated or separable in a fluid-tight manner from each other.

The disclosed device further comprises a pumping device for pumping a fluid from the outer reservoir into the inner reservoir so that the fluid in the inner reservoir generates a pressure from an outside direction on the wall of the bladder so that emptying of the bladder is supported. The pressure on the bladder wall is generated since the fluid is pumped into the inner reservoir, thereby displacing the bladder wall so that the bladder is forced to be emptied.

Thus, the disclosure provides for a hydraulically or pneumatically actuated device for emptying the bladder of a patient. This kind of actuation helps to preserve the tissue of the bladder while pressure is applied on the bladder wall. The idea of hydraulically supporting the function of other organs in the body of a patient is known e.g. from ventricular assist devices which support the function of the heart of a patient.

The disclosed device further has the advantage that the filling level of the bladder of a patient can be detected even after a surgery of the bladder. In a bladder surgery it is possible to enlarge the bladder volume by using bowel. A negative side effect after such a surgery is that the patient may lose the sensation of the bladder. The disclosed device allows detecting the filling level of the bladder even if the bladder volume is enlarged after a surgery and the patient has lost his sensation of the bladder. It is possible to use a surrounding element, which has a larger volume than the usual filling volume of the bladder so that by using the disclosed device the volume of the bladder can be increased.

It is preferred that the surrounding element comprises at least one port for transmitting fluid from the outer reservoir to the inner reservoir and vice versa. In a preferred embodiment this port comprises a valve. The pumping device is adapted to pump the fluid through this port from the outer reservoir to the inner reservoir. When the pumping direction of the pumping device is reversed, fluid will be pumped from the inner reservoir back to the outer reservoir so that after emptying of the bladder filling of the bladder can take place. When emptying the bladder by pumping fluid from the outer reservoir to the inner reservoir, the volume of fluid which is pumped during a certain time has to be controlled so that the bladder is not emptied too fast. Emptying the bladder too fast may lead to an increased pressure in the bladder causing a reflux of urine into the kidneys.

In the disclosed device it is important to detect the filling level of the bladder since, if the bladder is completely filled, urine may enter into the kidneys. Further, it may be important to detect that the bladder is completely empty since such a situation may cause an infection risk.

It is preferred that a defined volume of fluid is used meaning that the volume of the fluid will not change over time. By using an inelastic outer membrane it is possible to accurately control the pumping process. The fluid can be, e.g., a hydraulic fluid.

In a preferred embodiment, the surrounding element or a lower part of the surrounding element comprises a silk matrix having plastic coating laminated thereon.

It is further preferred that in this embodiment the silk matrix is not laminated at the lower end of the surrounding element in the area where the surrounding element is connectable to the bladder so that a connecting element comprising silk is formed in this area. This connecting element is preferred to be formed by connecting fibers comprising silk which may adhere with the bladder tissue. It is preferred that, during this adherence process, the bladder of the patient has a minimum filling level, meaning that it is not completely empty.

Using a silk material for the surrounding element has the advantage that the surrounding element has a high stability and resistance and at the same time can be easily connected by an adherence process to the bladder tissue without being rejected by the body of the patient.

It is preferred that the surrounding element is formed such that it is essentially stiff, meaning that it has a defined shape without needing further supporting elements for maintaining its shape. Depending on the stiffness of the surrounding element, the sensitivity of the sensors may be adapted if the sensors are placed at the inner side of the surrounding element.

It is further preferred that at least a part of the bladder wall comprises a plastics material which may be, e.g., a polyurethane material. This plastics material may be used either in addition to the natural bladder wall or as a replacement for at least a part of the natural bladder wall thus forming an artificial bladder wall. In a preferred embodiment the artificial bladder wall comprises a coating on its inner side. Such a coating serves for preventing crystallization of urine on the inner side of the artificial bladder wall.

It is further preferred that the sensor for detecting the filling level of the bladder is formed at the inner side of the surrounding element facing towards the bladder. A plurality of sensors, i.e. four sensors, may be used in this embodiment. The at least one sensor may be a pressure sensor, in particular a piezo sensor or a distance sensor, e.g. an optical or capacitive distance sensor or an ultrasound distance sensor.

As an alternative, the sensor may be formed by at least two electrodes which are placed in or on the bladder wall. These electrodes are adapted to measure the electrical resistance of the part of the bladder wall between them so that the filling level of the bladder can be derived from the extent of the stretching of the bladder wall between the electrodes. This stretching results in a change of the electrical resistance of this part of the bladder wall, whereby this stretching can be in the range of a few thousands of millimeters.

The disclosure further relates to a method for operating a device as it is described in the present application. The device has a flexible membrane which is inelastic. Further the device may comprise an artificial bladder wall, which is substantially inelastic. According to the disclosed method the motor current of the pump is measured. When the inner reservoir is full, meaning that no more fluid can be pumped into the inner reservoir, the motor current of the pump will rise. If a rise in the motor current of the pump is detected no more fluid is pumped from the outer reservoir into the inner reservoir.

An independent disclosure is related to a method for providing a surrounding element for a bladder, in particular for use in a device as it is described in the present application. First, a dome-shaped silk matrix is provided. A plastics coating which may be a polyurethane coating is laminated onto an upper part of the silk matrix, hereby omitting a lower end of the silk matrix, thereby forming a coating-free area at the lower end of the dome-shaped surrounding element. This coating-free area may be used as an adhering area for connecting the surrounding element to the bladder of a patient.

### BRIEF DESCRIPTION OF THE DRAWING

FIG. 1 is a schematic representation of a device according to the present disclosure.

### DETAILED DESCRIPTION OF THE PREFERRED EMBODIMENT

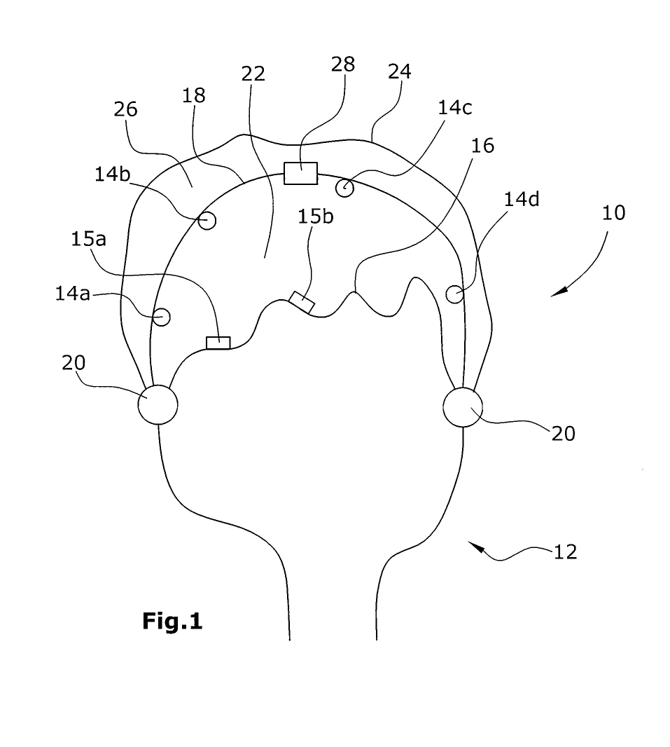

A preferred embodiment of the disclosure is now described in the context of FIG. 1.

Bladder 12 is connected to an artificial bladder wall 16 by connecting elements 20 which may be formed from silk fibers in the lower end of surrounding element 18 and the artificial bladder wall 16. The surrounding element 18 surrounds the bladder wall 16. In a first embodiment four pressure sensors 14a-14d are placed at the inner side of the surrounding element 18 facing towards the bladder 12. If the bladder 12 is completely filled or has reached a pre-defined filling level, the bladder wall 16 will contact at least one of the pressure sensors 14a-14d so that the filling level of the bladder 12 can be automatically detected.

Then, by a pump which is not shown fluid will be pumped from the outer reservoir 26 which is defined in the hollow space between the surrounding element 18 and the flexible membrane 24 into the inner reservoir 22 which is defined between the bladder wall 16 and the surrounding element 18. The transport of the fluid from the outer reservoir 26 into the inner reservoir 22 will take place through port 28 which is formed in the surrounding element 18.

In order to allow the refilling process of the bladder, fluid will be pumped back from the inner reservoir 22 into the outer reservoir 26 after the bladder has been emptied.

In an alternative embodiment, electrodes 15a, 15b may be used instead of pressure sensors 14a-14d. These electrodes 15a, 15b are placed in a certain distance to each other in or on the bladder wall 16 and are adapted to measure the electrical resistance of the part of the bladder wall 16 which is arranged between them. Depending on the filling level of the bladder 12, this part of the bladder wall 16 will be stretched so that its electrical resistance will change. Thus, the filling level of the bladder 12 can be detected by electrodes 15a, 15b.

What is claimed is:

1. Device for supporting the emptying of a bladder of a patient, the device comprising:
an essentially stiff dome-shaped surrounding element for surrounding at least a part of the bladder having a variable volume,
the device comprising at least one sensor for detecting the filling level of the bladder,
the surrounding element being connectable with the bladder at a lower end of the surrounding element in a fluid-tight manner, so that an inner reservoir is defined in a hollow space between the surrounding element and the wall of the bladder,
the device further comprising a flexible membrane surrounding the surrounding element, the flexible membrane being connectable with the bladder in a fluid-tight manner, so that an outer reservoir is defined in a hollow space between the flexible membrane and the surrounding element,
the device further comprising a pumping device for pumping a fluid from the outer reservoir into the inner reservoir so that the fluid in the inner reservoir generates a pressure from an outside direction on the wall of the bladder so that emptying of the bladder is supported.

2. Device according to claim 1, wherein the surrounding element comprises at least one port for transmitting fluid from the outer reservoir to the inner reservoir and vice versa.

3. Device according to claim 2, wherein the surrounding element comprises a silk matrix having a plastic coating laminated thereon.

4. Device according to claim 3, wherein the silk matrix is not laminated at the lower end of the surrounding element in the area where the surrounding element is connectable to the bladder so that a connecting element, in particular connecting fibers comprising silk, are formed in this area which may adhere with the bladder tissue.

5. Device according to claim 1, wherein at least a part of the bladder wall comprises a plastics material, in particular a polyurethane material either in addition to the natural bladder wall or as a replacement for at least a part of the natural bladder wall thus forming an artificial bladder wall.

6. Device according to claim 5, characterized in that the artificial bladder wall comprises a coating on its inner side, the coating serving for preventing crystallization of urine on the inner side of the artificial bladder wall.

7. Device according to claim 1, wherein the sensor is formed at the inner side of the surrounding element facing towards the bladder and this sensor is a pressure sensor, in particular a piezo sensor or a distance sensor, in particular an optical or capacitive distance sensor or an ultra sound distance sensor.

8. Device according to claim 1, wherein the sensor is formed by at least two electrodes placed in or on a bladder wall, the electrodes being adapted to measure the electrical resistance of the part of the bladder wall between them, so that the filling level of the bladder can be derived from the extent of the stretching of the bladder wall between the electrodes which results in a change of the electrical resistance of this part of the bladder wall.

9. Method for operating a device comprising: an essentially stiff dome-shaped surrounding element for surrounding at least a part of the bladder having a variable volume, the device comprising at least one sensor for detecting the filling level of the bladder, the surrounding element being connectable with the bladder at a lower end of the surrounding element in a fluid-tight manner, so that an inner reservoir is defined in a hollow space between the surrounding element and the wall of the bladder, the device further comprising a flexible membrane surrounding the surrounding element, the flexible membrane being connectable with the bladder in a fluid-tight manner, so that an outer reservoir is defined in a hollow space between the flexible membrane and the surrounding element, the device further comprising a pumping device for pumping a fluid from the outer reservoir into the inner reservoir so that the fluid in the inner reservoir generates a pressure from an outside direction on the wall of the bladder so that emptying of the bladder is supported, wherein the flexible membrane is inelastic, wherein the method comprises:
measuring the motor current of the pump; and
pumping the fluid from the outer reservoir into the inner reservoir until a rise in the motor current of the pump is detected, meaning that due to the inelastic membrane no more fluid can be pumped into the inner reservoir.

10. Method for producing a surrounding element for a bladder, the method comprising:
providing a dome-shaped silk matrix, and laminating a plastics coating, in particular a polyurethane coating onto an upper part of the silk matrix, thereby omitting a lower end of the silk matrix and forming a coating-free area at the lower end of the dome-shaped surrounding element.
